(19) United States
(12) Patent Application Publication
(10) Pub. No.: US 2008/0077248 A1
(43) Pub. Date: Mar. 27, 2008

(54) Vertebral body replacement

(75) Inventors: Fred Murillo, San Diego, CA (US); Laurence Mercer McKinley, Escondido, CA (US)

Correspondence Address: Brian P. Hopkins;Mintz Levin Cohn Ferris Glovsky and Popeo PC, Chrysler Center, 666 Third Avenue, New York, NY, 10017, US

(73) Assignee: ALPHATEC SPINE, INC., Carlsbad, CA (US)

(21) Appl. No.: 11/903,938

(22) Filed: Sep. 24, 2007

Related U.S. Application Data
(60) Provisional application No. 60/846,474, filed on Sep. 22, 2006.

(51) Int. Cl. A61F 2/44

(52) U.S. Cl. 623/17.16

(57) ABSTRACT

A spacer for use in spine fusion surgical procedures is disclosed. The spacer includes an enclosure having a wall that is configured to enclose a hollow interior. The wall is further configured to include a plurality of openings spaced throughout the wall. The openings are configured to connect an exterior of the enclosure to the hollow interior. The enclosure further includes an indication cutting line configured to allow adjustment of a height of the enclosure.

### CROSS-REFERENCE TO RELATED APPLICATIONS

The present application claims priority to U.S. Provisional Patent Application No. 60/846,474 to Murillo et al., filed Sep. 22, 2006, entitled “Titanium Mesh Vertebral Body Replacement”, and incorporates its disclosure herein by reference in its entirety.

### BACKGROUND OF THE INVENTION

1. Field of the Invention

The present invention is directed to systems, methods, and devices applicable to spinal surgery. More specifically, the present invention relates to spine fusion procedures. Specifically, the present invention relates to a vertebral body replacement assembly.

2. Background of the Invention

Vertebrae are the individual irregular bones that make up the spinal column (aka ischis)—a flexuous and flexible column. There are normally thirty-three vertebrae in humans, including the five that are fused to form the sacrum (the others are separated by intervertebral discs) and the four coccygeal bones which form the tailbone. The upper three regions comprise the remaining 24, and are grouped under the names cervical (7 vertebrae), thoracic (12 vertebrae) and lumbar (5 vertebrae), according to the regions they occupy. This number is sometimes increased by an additional vertebra in one region, or it may be diminished in one region, the deficiency often being supplied by an additional vertebra in another. The number of cervical vertebrae is, however, very rarely increased or diminished.

A typical vertebra consists of two essential parts: an anterior (front) segment, which is the vertebral body; and a posterior part—the vertebral (neural) arch—which encloses the vertebral foramen. The vertebral arch is formed by a pair of pedicles and a pair of laminae, and supports seven processes, four articular, two transverse, and one spinous, the latter also being known as the neural spine.

When the vertebrae are articulated with each other, the bodies form a strong pillar for the support of the head and trunk, and the vertebral foramina constitute a canal for the protection of the medulla spinalis (spinal cord), while between every pair of vertebrae are two apertures, the intervertebral foramina, one on either side, for the transmission of the spinal nerves and vessels.

Conventional systems for vertebral body replacement are used in spinal fusion procedures to repair damaged or incorrectly articulating vertebrae. Spinal fusion employs the use of spacer assemblies having a hollow mesh spacer tube and end caps that space apart and fuse together adjacent vertebrae. These mesh spacer tubes are often formed of titanium and are available in varying shapes and sizes. In addition, they can be trimmed on site by the surgeon to provide a better individual fit for each patient. Conventional spinal spacer assemblies come in different cross sections. These spacer assemblies are generally hollow and include openings in the side thereof to provide access for bone to grow and fuse within the mesh tube.

There exists a need for further improvements in the field of vertebral body replacement assemblies of the present type.

### SUMMARY OF THE INVENTION

In some embodiments, the present invention relates to a titanium mesh vertebral spacer that can be used with the Transforaminal Lumbar Interbody Fusion (“TLIF”) and Posterior Lumbar Interbody Fusion (“PLIF”) instruments for an initial discectomy. The spacer can be configured to fit in an anterior portion of the body. The spacer can have variable cross-section. The cross-section can be circular, oval, or other desired shape. Further, the spacer can also include a variable shape mesh pattern. The pattern can consist of circles, ovals, squares, rectangles, polygons, ellipses or other shapes.

In an embodiment, the wall of the spacer mesh has a 1.6 mm wall thickness. In an embodiment, the spacer can include an indication on the outer side of the wall for cutting the spacer.

In some embodiments, the present invention relates to a spacer for use in spine fusion surgical procedures. The spacer includes an enclosure having a wall that is configured to enclose a hollow interior. The wall is further configured to include a plurality of openings spaced throughout the wall. The openings are configured to connect an exterior of the enclosure to the hollow interior. The enclosure further includes an indication cutting line configured to allow adjustment of a height of the enclosure.

In some embodiments, the present invention relates to a spinal vertebral replacement assembly. The assembly includes a spacer having an enclosure having a wall that is configured to enclose a hollow interior. The wall is further configured to include a plurality of openings spaced throughout the wall. The openings are configured to connect an exterior of the enclosure to the hollow interior. The enclosure further includes an indication cutting line configured to allow adjustment of a height of the enclosure.

Further features and advantages of the invention, as well as structure and operation of various embodiments of the invention, are disclosed in detail below with references to the accompanying drawings.

### BRIEF DESCRIPTION OF THE DRAWINGS

The present invention is described with reference to the accompanying drawings. In the drawings, like reference numbers indicate identical or functionally similar elements.

FIGS. 1A-1D are prospective views of an exemplary vertebral body replacement assembly, according to embodiments of the present invention.

FIG. 1E is a top view of the exemplary vertebral body replacement assembly shown in FIGS. 1A-1D.

FIG. 1F is a detailed view of a portion of the exemplary vertebral body replacement assembly shown in FIGS. 1A-1D.

FIGS. 1G-1J are side views of exemplary vertebral body replacement assembly shown in FIGS. 1A-1D.

FIGS. 2A-2D are prospective views of another exemplary vertebral body replacement assembly, according to embodiments of the present invention.

FIG. 2E is a top view of the exemplary vertebral body replacement assembly shown in FIGS. 2A-2D.

FIG. 2F is a detailed view of a portion of the exemplary vertebral body replacement assembly shown in FIGS. 2A-2D.

FIGS. 2G-2J are side views of exemplary vertebral body replacement assembly shown in FIGS. 2A-2D.

FIGS. 3A-3D are prospective views of yet another exemplary vertebral body replacement assembly, according to embodiments of the present invention.

FIG. 3E is a top view of the exemplary vertebral body replacement assembly shown in FIGS. 3A-3D.

FIG. 3F is a detailed view of a portion of the exemplary vertebral body replacement assembly shown in FIGS. 3A-3D.

FIGS. 3G-3J are side views of exemplary vertebral body replacement assembly shown in FIGS. 3A-3D.

FIGS. 4A-4D are prospective views of yet another exemplary vertebral body replacement assembly, according to embodiments of the present invention.

FIG. 4E is a top view of the exemplary vertebral body replacement assembly shown in FIGS. 4A-4D.

FIG. 4F is a detailed view of a portion of the exemplary vertebral body replacement assembly shown in FIGS. 4A-4D.

FIGS. 4G-4J are side views of exemplary vertebral body replacement assembly shown in FIGS. 4A-4D.

FIGS. 5A-5D are prospective views of yet another exemplary vertebral body replacement assembly, according to embodiments of the present invention.

FIG. 5E is a top view of the exemplary vertebral body replacement assembly shown in FIGS. 5A-5D.

FIG. 5F is a detailed view of a portion of the exemplary vertebral body replacement assembly shown in FIGS. 5A-5D.

FIGS. 5G-5J are side views of exemplary vertebral body replacement assembly shown in FIGS. 5A-5D.

FIGS. 6A-6D are prospective views of yet another exemplary vertebral body replacement assembly, according to embodiments of the present invention.

FIG. 6E is a top view of the exemplary vertebral body replacement assembly shown in FIGS. 6A-6D.

FIG. 6F is a detailed view of a portion of the exemplary vertebral body replacement assembly shown in FIGS. 6A-6D.

FIGS. 6G-6J are side views of exemplary vertebral body replacement assembly shown in FIGS. 6A-6D.

FIGS. 7A-7D are prospective views of yet another exemplary vertebral body replacement assembly, according to embodiments of the present invention.

FIG. 7E is a top view of the exemplary vertebral body replacement assembly shown in FIGS. 7A-7D.

FIG. 7F is a detailed view of a portion of the exemplary vertebral body replacement assembly shown in FIGS. 7A-7D.

FIGS. 7G-7J are side views of exemplary vertebral body replacement assembly shown in FIGS. 7A-7D.

FIGS. 8A-8D are prospective views of yet another exemplary vertebral body replacement assembly, according to embodiments of the present invention.

FIG. 8E is a top view of the exemplary vertebral body replacement assembly shown in FIGS. 8A-8D.

FIG. 8F is a detailed view of a portion of the exemplary vertebral body replacement assembly shown in FIGS. 8A-8D.

FIGS. 8G-8J are side views of exemplary vertebral body replacement assembly shown in FIGS. 8A-8D.

FIGS. 9A-9D are prospective views of yet another exemplary vertebral body replacement assembly, according to embodiments of the present invention.

FIG. 9E is a top view of the exemplary vertebral body replacement assembly shown in FIGS. 9A-9D.

FIG. 9F is a detailed view of a portion of the exemplary vertebral body replacement assembly shown in FIGS. 9A-9D.

FIGS. 9G-9J are side views of exemplary vertebral body replacement assembly shown in FIGS. 9A-9D.

### DETAILED DESCRIPTION OF THE INVENTION

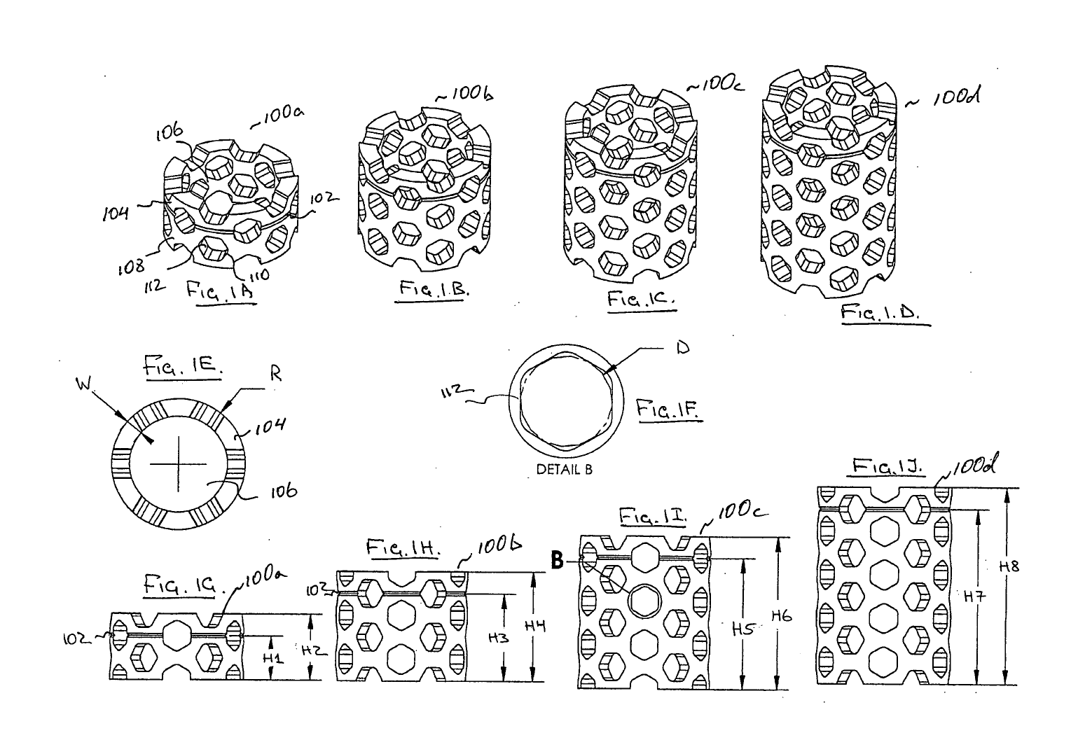

The present invention relates to spinal fusion procedures and surgeries. In particular, the present invention relates to a vertebral body replacement assembly. FIGS. 1A-9J illustrate various embodiments of the vertebral body replacement assembly, which will be also referred to as a spacer. Such reference is for ease of description and is not intended to limit the scope of the invention.

FIGS. 1A-1J illustrate exemplary embodiments of the spacers 100(a, b, c, d) that include a plurality of openings that can be arranged in a mesh pattern. As illustrated in these embodiments, spacers 100 have an outer diameter R. In some embodiments, R=12 mm. As can be understood by one skilled in the art, other diameters of the spacers 100 are possible.

FIGS. 1A-1D are perspective, cross-sectional views of variable height spacers 100 (a, b, c, d). The spacer 100 includes a wall 104 having a thickness W. As shown in of FIG. 1E, which is a top view of the spacers 100, the thickness W can be 1.6 mm. The wall 104 encloses a hollow interior 106 and also includes an exterior 108. Each of the embodiments in FIGS. 1A-1D include an indication cutting line 102 on the exterior 108, where the cutting line 102 is located towards the top of the spacers 100. As can be understood by one skilled in the art, the cutting line 102 can be located anywhere on the outer wall of the spacer 100. Further, there can be more than one indication cutting line on the spacers 100. The cutting line 102 can be configured to allow a surgeon (or other medical personnel, technician, etc.) to adjust the height of the spacer 100 either prior to installation of the spacer 100 or subsequent to installation of the spacer. In some embodiments, the cutting line 102 can be configured to be an indentation in the exterior 108 of the wall 104. The cutting line 102 can be configured to connect openings 112, as illustrated in FIGS. 1A-1D and 1G-1J. This allows a surgeon (or any other authorized medical personnel) to evenly cut and adjust the spacer 100 to a specific height.

The wall 104 further includes a mesh pattern 110 that consists of variable-shaped openings 112 that extend from the exterior 108 to the hollow interior 106 of the spacer 100. The opening 112 is shown in more detail in FIG. 1F. In the embodiment of FIGS. 1A-1J, the opening 112 has a hexagonal shape. In this embodiment, the distance D from the center of the opening 112 to one of its sides is approximately 2.5 mm. FIGS. 1G-1J are side views of variable-height spacers 100. As shown in FIG. 1G, spacer 100a has a total height H2 and a height H1 to the indication cutting line 102. In some embodiments H1=4 mm, H2=6 mm. As shown in FIG. 1H, spacer 100b has a total height H4 and a height H3 to the indication cutting line 102. In some embodiments H3=8 mm, H4=10 mm. As shown in FIG. 1I, spacer 100c has a total height H6 and a height H5 to the indication cutting line 102. In some embodiments H5=12 mm, H2=14 mm. As shown in FIG. 1J, spacer 100d has a total height H8 and a height H7 to the indication cutting line 102. In some embodiments H7=16 mm, H2=18 mm. As shown in FIGS. 1G-1J, the total heights of the spacers 100 range from 6 mm to 18 mm (as shown in FIGS. 1G-1J, the height of the spacers 100 increases in 4 mm increments). Also, as shown in embodiments of FIGS. 1G-1J, the cutting line 102 is located 2 mm from the top of the spacers 100. As can be understood by one skilled in the art, the spacers 100 have variable diameters, heights, shapes of the mesh pattern, and thickness.

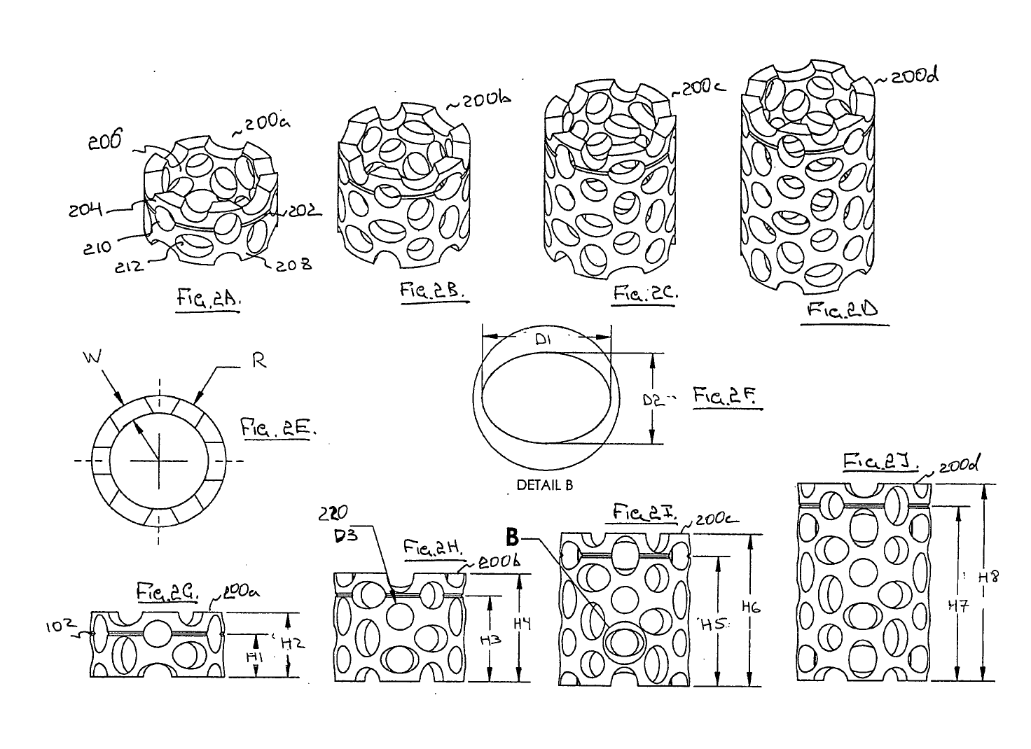

FIGS. 2A-2J illustrate exemplary embodiments of the spacers 200(a, b, c, d) that include a plurality of openings that can be arranged in a mesh pattern. As illustrated in these embodiments, spacers 200 have an outer diameter R. In some embodiments, R=12 mm. As can be understood by one skilled in the art, other diameters of the spacers 200 are possible.

FIGS. 2A-2D are perspective, cross-sectional views of variable height spacers 200 (a, b, c, d). The spacer 200 includes a wall 204 having a thickness W. As shown in of FIG. 2E, which is a top view of the spacers 200, the thickness W can be 1.6 mm. The wall 204 encloses a hollow interior 206 and also includes an exterior 208. Each of the embodiments in FIGS. 2A-2D include an indication cutting line 202 on the exterior 208, where the cutting line 202 is located towards the top of the spacers 200. As can be understood by one skilled in the art, the cutting line 202 can be located anywhere on the outer wall of the spacer 200. Further, there can be more than one indication cutting line on the spacers 200. The cutting line 202 can be configured to allow a surgeon (or other medical personnel, technician, etc.) to adjust the height of the spacer 200 either prior to installation of the spacer 200 or subsequent to installation of the spacer. In some embodiments, the cutting line 202 can be configured to be an indentation in the exterior 208 of the wall 204. The cutting line 202 can be configured to connect openings 212 and 220, as illustrated in FIGS. 2A-2D and 2G-2J. This allows a surgeon (or any other authorized medical personnel) to evenly cut and adjust the spacer 200 to a specific height.

The wall 204 further includes a mesh pattern 210 that consists of variable-shaped openings 210 and 220 that extend from the exterior 208 to the hollow interior 206 of the spacer 200. The opening 212 is shown in more detail in FIG. 2F. In the embodiment of FIGS. 2A-2J, the opening 212 has an oval shape. In this embodiment, the first diameter D1 of the oval shaped opening 212 is approximately 3.5 mm and the second diameter D2 of the oval shaped opening 212 is approximately 2.5 mm. The opening 220 has a round shape with a diameter D3. In some embodiments, D3 is equal to 2.5 mm. FIGS. 2G-2J are side views of variable-height spacers 200. As shown in FIG. 2G, spacer 200a has a total height H1 and a height H2 to the indication cutting line 202. In some embodiments, H1=4 mm, H2=6 mm. As shown in FIG. 2H, spacer 200b has a total height H4 and a height H3 to the indication cutting line 202. In some embodiments H3=8 mm, H4=10 mm. As shown in FIG. 2I, spacer 200c has a total height H6 and a height H5 to the indication cutting line 202. In some embodiments, H5=12 mm, H6=14 mm. As shown in FIG. 2J, spacer 200d has a total height H8 and a height H7 to the indication cutting line 202. In some embodiments, H7=16 mm, H8=18 mm. As shown in FIGS. 2G-2J, the total heights of the spacers 200 range from 6 mm to 18 mm (as shown in FIGS. 2G-2J, the height of the spacers 200 increases in 4 mm increments). Also, as shown in embodiments of FIGS. 2G-2J, the cutting line 202 is located 2 mm from the top of the spacers 200. As can be understood by one skilled in the art, the spacers 200 have variable diameters, heights, shapes of the mesh pattern, and thickness.

Additionally, the oval-shaped openings 212 can be aligned in different directions as shown in FIGS. 2A-2D and 2G-2J. Also, some of the openings 212 (or 220) can be circular or any other shape. The openings 212 can have a diameter that varies from the exterior 208 to the interior 206. The oval shaped openings 212 and the circular openings 220 can be arranged in a pattern as illustrated in FIGS. 2A-2D and 2G-2J. Further, one opening 212 can be perpendicularly arranged to the other opening 212. Alternatively, the openings 212 can be arranged at different angles with regard to each other.

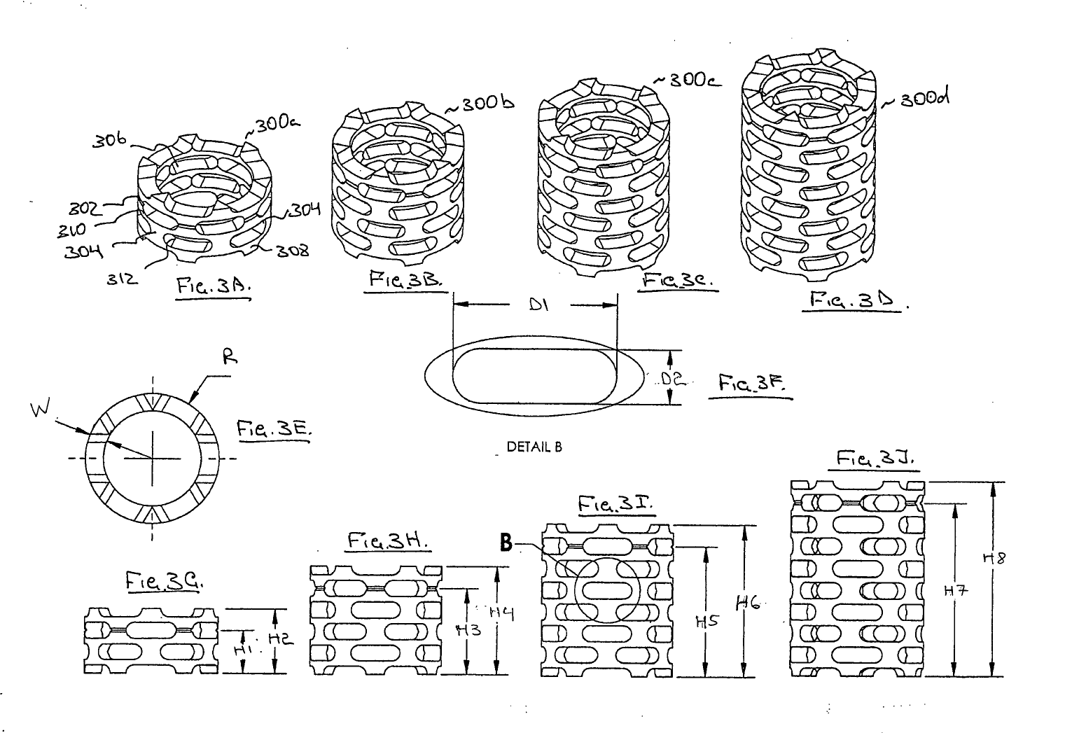

FIGS. 3A-3J illustrate exemplary embodiments of the spacers 300(a, b, c, d) that include a plurality of openings that can be arranged in a mesh pattern. As illustrated in these embodiments, spacers 300 have an outer diameter R. In some embodiments, R=12 mm. As can be understood by one skilled in the art, other diameters of the spacers 300 are possible.

FIGS. 3A-3D are perspective, cross-sectional views of variable height spacers 300 (a, b, c, d). The spacer 300 includes a wall 304 having a thickness W. As shown in of FIG. 3E, which is a top view of the spacers 300, the thickness W can be 1.6 mm. The wall 304 encloses a hollow interior 306 and also includes an exterior 308. Each of the embodiments in FIGS. 3A-3D include an indication cutting line 302 on the exterior 308, where the cutting line 302 is located towards the top of the spacers 300. As can be understood by one skilled in the art, the cutting line 302 can be located anywhere on the outer wall of the spacer 300. Further, there can be more than one indication cutting line on the spacers 300. The cutting line 302 can be configured to allow a surgeon (or other medical personnel, technician, etc.) to adjust the height of the spacer 300 either prior to installation of the spacer 300 or subsequent to installation of the spacer. In some embodiments, the cutting line 302 can be configured to be an indentation in the exterior 308 of the wall 304. The cutting line 302 can be configured to connect openings 312, as illustrated in FIGS. 3A-3D and 3G-3J. This allows a surgeon (or any other authorized medical personnel) to evenly cut and adjust the spacer 300 to a specific height.

The wall 304 further includes a mesh pattern 310 that consists of variable-shaped openings 312 that extend from the exterior 308 to the hollow interior 306 of the spacer 300. The opening 312 is shown in more detail in FIG. 3F. In the embodiment of FIGS. 3A-3J, the opening 312 has an elliptical shape. In this embodiment, the first diameter D1 of the elliptical shape opening 312 is approximately 4.5 mm and the second diameter D2 of the elliptical shape opening 312 is approximately 1.5 mm. FIGS. 3G-3J are side views of variable-height spacers 300. As shown in FIG. 3G, spacer 300a has a total height H2 and a height H1 to the indication cutting line 302. In some embodiments, H1=4 mm, H2=6 mm. As shown in FIG. 3H, spacer 300b has a total height H4 and a height H3 to the indication cutting line 302. In some embodiments, H3=8 mm, H4=10 mm. As shown in FIG. 31, spacer 300c has a total height H6 and a height H5 to the indication cutting line 302. In some embodiments, H5=12 mm, H6=14 mm. As shown in FIG. 3J, spacer 300d has a total height H8 and a height H7 to the indication cutting line 302. In some embodiments, H7=16 mm, H8=18 mm. As shown in FIGS. 3G-3J, the total heights of the spacers 300 range from 6 mm to 18 mm (as shown in FIGS. 3G-3J, the height of the spacers 300 increases in 4 mm increments). Also, as shown in embodiments of FIGS. 3G-3J, the cutting line 302 is located 2 mm from the top of the spacers 300. As can be understood by one skilled in the art, the spacers 300 have variable diameters, heights, shapes of the mesh pattern, and thickness.

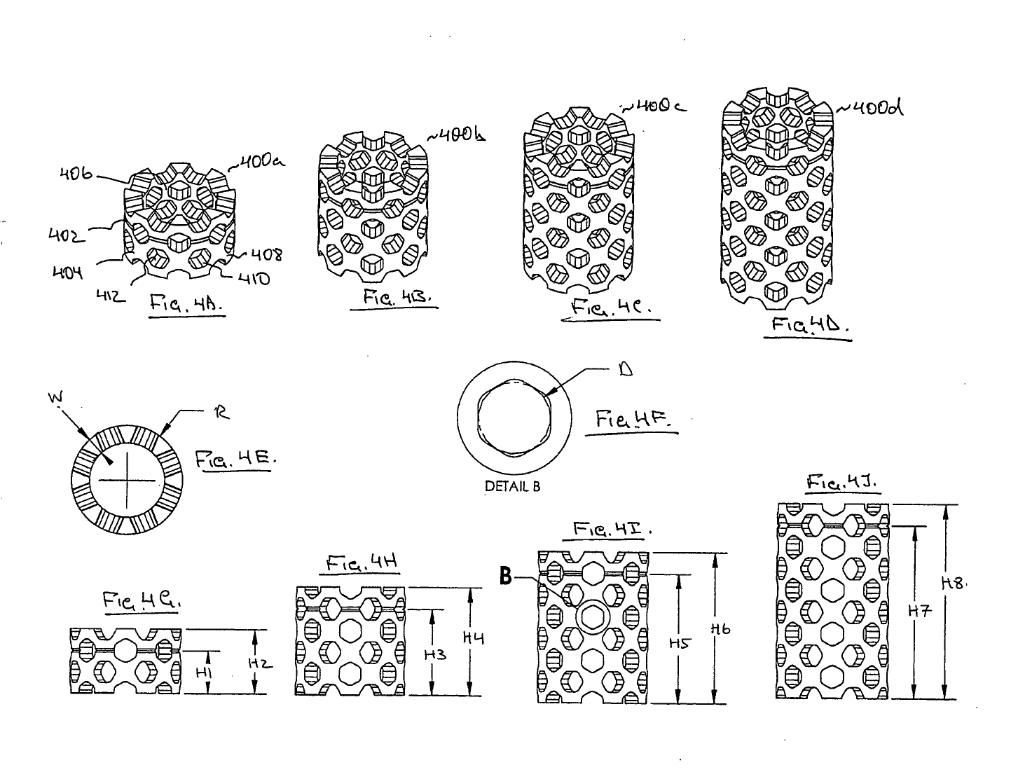

FIGS. 4A-4J illustrate exemplary embodiments of the spacers 400(a, b, c, d) that include a plurality of openings that can be arranged in a mesh pattern. As illustrated in these embodiments, spacers 400 have an outer diameter R. In some embodiments, R=10 mm. As can be understood by one skilled in the art, other diameters of the spacers 400 are possible.

FIGS. 4A-4D are perspective, cross-sectional views of variable height spacers 400 (a, b, c, d). The spacer 400 includes a wall 404 having a thickness W. As shown in of FIG. 4E, which is a top view of the spacers 400, the thickness W can be 1.6 mm. The wall 404 encloses a hollow interior 406 and also includes an exterior 408. Each of the embodiments in FIGS. 4A-4D include an indication cutting line 402 on the exterior 408, where the cutting line 402 is located towards the top of the spacers 400. As can be understood by one skilled in the art, the cutting line 402 can be located anywhere on the outer wall of the spacer 400. Further, there can be more than one indication cutting line on the spacers 400. The cutting line 402 can be configured to allow a surgeon (or other medical personnel, technician, etc.) to adjust the height of the spacer 400 either prior to installation of the spacer 400 or subsequent to installation of the spacer. In some embodiments, the cutting line 402 can be configured to be an indentation in the exterior 408 of the wall 404. The cutting line 402 can be configured to connect openings 412, as illustrated in FIGS. 4A-4D and 4G-4J. This allows a surgeon (or any other authorized medical personnel) to evenly cut and adjust the spacer 400 to a specific height.

The wall 404 further includes a mesh pattern 410 that consists of variable-shaped openings 412 that extend from the exterior 408 to the hollow interior 406 of the spacer 400. The opening 412 is shown in more detail in FIG. 4F. In the embodiment of FIGS. 4A-4J, the opening 412 has a hexagonal shape. In this embodiment, the distance D from the center of the opening 412 to one of its sides is approximately 2.0 mm. FIGS. 4G-4J are side views of variable-height spacers 400. As shown in FIG. 4G, spacer 400a has a total height H2 and a height H1 to the indication cutting line 402. In some embodiments, H1=4 mm, H2=6 mm. As shown in FIG. 4H, spacer 400b has a total height H4 and a height H3 to the indication cutting line 402. In some embodiments, H3=8 mm, H4=10 mm. As shown in FIG. 4I, spacer 400c has a total height H6 and a height H5 to the indication cutting line 402. In some embodiments, H5=12 mm, H6=14 mm. As shown in FIG. 4J, spacer 400d has a total height H8 and a height H7 to the indication cutting line 402. In some embodiments, H7=16 mm, H8=18 mm. As shown in FIGS. 4G-4J, the total heights of the spacers 400 range from 6 mm to 18 mm (as shown in FIGS. 4G-4J, the height of the spacers 400 increases in 4 mm increments). Also, as shown in embodiments of FIGS. 4G-4J, the cutting line 402 is located 2 mm from the top of the spacers 400. As can be understood by one skilled in the art, the spacers 400 have variable diameters, heights, shapes of the mesh pattern, and thickness.

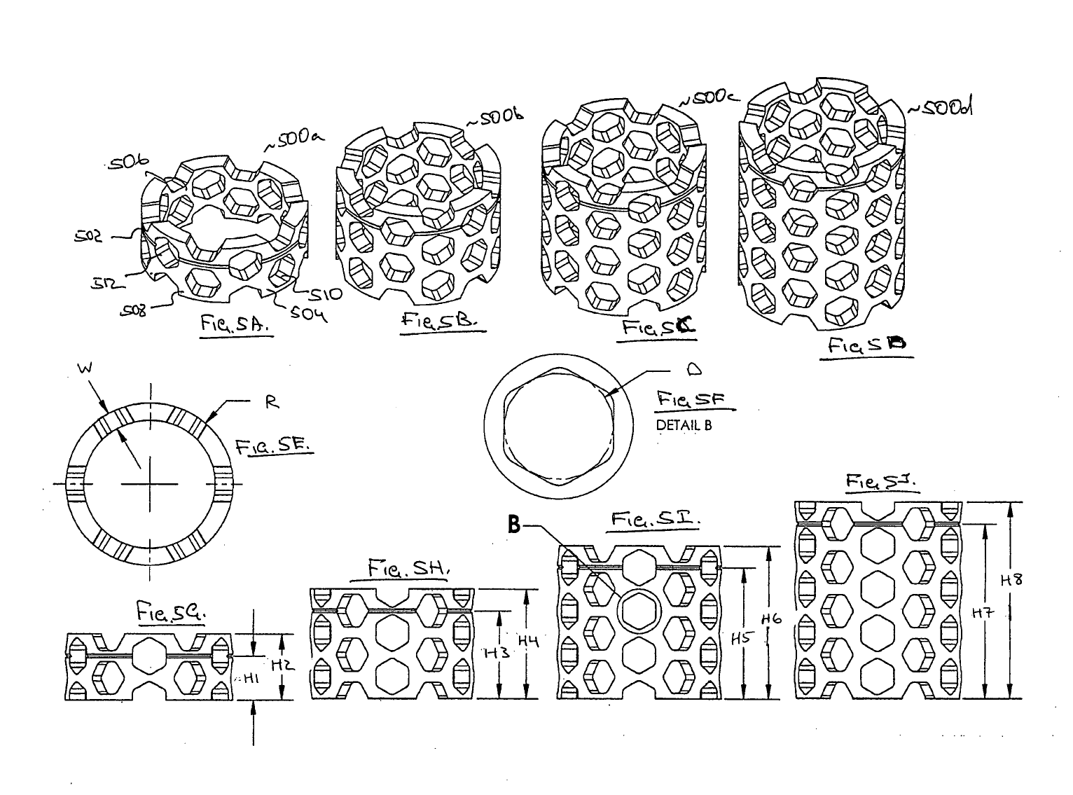

FIGS. 5A-5J illustrate exemplary embodiments of the spacers 500(a, b, c, d) that include a plurality of openings that can be arranged in a mesh pattern. As illustrated in these embodiments, spacers 500 have an outer diameter R. In some embodiments, R=15 mm. As can be understood by one skilled in the art, other diameters of the spacers 500 are possible.

FIGS. 5A-5D are perspective, cross-sectional views of variable height spacers 500 (a, b, c, d). The spacer 500 includes a wall 504 having a thickness W. As shown in of FIG. 5E, which is a top view of the spacers 500, the thickness W can be 1.6 mm. The wall 504 encloses a hollow interior 506 and also includes an exterior 508. Each of the embodiments in FIGS. 5A-5D include an indication cutting line 502 on the exterior 508, where the cutting line 502 is located towards the top of the spacers 500. As can be understood by one skilled in the art, the cutting line 502 can be located anywhere on the outer wall of the spacer 500. Further, there can be more than one indication cutting line on the spacers 500. The cutting line 502 can be configured to allow a surgeon (or other medical personnel, technician, etc.) to adjust the height of the spacer 500 either prior to installation of the spacer 500 or subsequent to installation of the spacer. In some embodiments, the cutting line 502 can be configured to be an indentation in the exterior 508 of the wall 504. The cutting line 502 can be configured to connect openings 512, as illustrated in FIGS. 5A-5D and 5G-5J. This allows a surgeon (or any other authorized medical personnel) to evenly cut and adjust the spacer 500 to a specific height.

The wall 504 further includes a mesh pattern 510 that consists of variable-shaped openings 512 that extend from the exterior 508 to the hollow interior 506 of the spacer 500. The opening 512 is shown in more detail in FIG. 5F. In the embodiment of FIGS. 5A-5J, the opening 512 has a hexagonal shape. In this embodiment, the distance D from the center of the opening 552 to one of its sides is approximately 3.0 mm. FIGS. 5G-5J are side views of variable-height spacers 500. As shown in FIG. 5G, spacer 500a has a total height H2 and a height H1 to the indication cutting line 502. In some embodiments H1=4 mm, H2=6 mm. As shown in FIG. 5H, spacer 500b has a total height H4 and a height H3 to the indication cutting line 502. In some embodiments, H3=8 mm, H4=10 mm. As shown in FIG. 5I, spacer 500c has a total height H6 and a height H5 to the indication cutting line 502. In some embodiments, H5=12 mm, H6=14 mm. As shown in FIG. 5J, spacer 500d has a total height H8 and a height H7 to the indication cutting line 502. In some embodiments, H7=16 mm, H8=18 mm. As shown in FIGS. 5G-5J, the total heights of the spacers 500 range from 6 mm to 18 mm (as shown in FIGS. 5G-5J, the height of the spacers 500 increases in 4 mm increments). Also, as shown in embodiments of FIGS. 5G-5J, the cutting line 502 is located 2 mm from the top of the spacers 500. As can be understood by one skilled in the art, the spacers 500 have variable diameters, heights, shapes of the mesh pattern, and thickness.

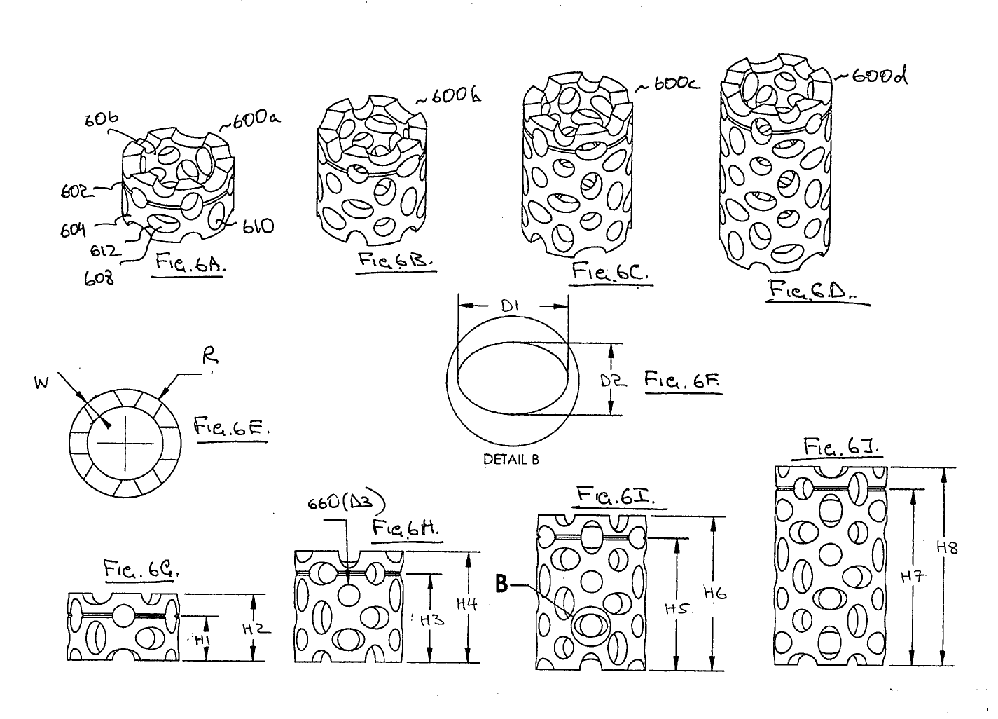

FIGS. 6A-6J illustrate exemplary embodiments of the spacers 600(a, b, c, d) that include a plurality of openings that can be arranged in a mesh pattern. As illustrated in these embodiments, spacers 600 have an outer diameter R. In some embodiments, R=10 mm. As can be understood by one skilled in the art, other diameters of the spacers 600 are possible.

FIGS. 6A-6D are perspective, cross-sectional views of variable height spacers 600 (a, b, c, d). The spacer 600 includes a wall 604 having a thickness W. As shown in of FIG. 6E, which is a top view of the spacers 600, the thickness W can be 1.6 mm. The wall 604 encloses a hollow interior 606 and also includes an exterior 608. Each of the embodiments in FIGS. 6A-6D include an indication cutting line 602 on the exterior 608, where the cutting line 602 is located towards the top of the spacers 600. As can be understood by one skilled in the art, the cutting line 602 can be located anywhere on the outer wall of the spacer 600. Further, there can be more than one indication cutting line on the spacers 600. The cutting line 602 can be configured to allow a surgeon (or other medical personnel, technician, etc.) to adjust the height of the spacer 600 either prior to installation of the spacer 600 or subsequent to installation of the spacer. In some embodiments, the cutting line 602 can be configured to be an indentation in the exterior 608 of the wall 604. The cutting line 602 can be configured to connect openings 612 and 660, as illustrated in FIGS. 6A-6D and 6G-6J. This allows a surgeon (or any other authorized medical personnel) to evenly cut and adjust the spacer 600 to a specific height.

The wall 604 further includes a mesh pattern 610 that consists of variable-shaped openings 610 and 660 that extend from the exterior 608 to the hollow interior 606 of the spacer 600. The opening 612 is shown in more detail in FIG. 6F. In the embodiment of FIGS. 6A-6J, the opening 612 has an oval shape. In this embodiment, the first diameter D1 of the oval shaped opening 612 is approximately 3.0 mm and the second diameter D2 of the oval shaped opening 612 is approximately 2.0 mm. The opening 660 has a round shape with a diameter D3. In some embodiments, D3 is equal to 2.0 mm. FIGS. 6G-6J are side views of variable-height spacers 600. As shown in FIG. 6G, spacer 600a has a total height H1 and a height H2 to the indication cutting line 602. In some embodiments, H1=4 mm, H2=6 mm. As shown in FIG. 6H, spacer 600b has a total height H4 and a height H3 to the indication cutting line 602. In some embodiments, H3=8 mm, H4=10 mm. As shown in FIG. 6I, spacer 600c has a total height H6 and a height H5 to the indication cutting line 602. In some embodiments, H5=12 mm, H6=14 mm. As shown in FIG. 6J, spacer 600d has a total height H8 and a height H7 to the indication cutting line 602. In some embodiments, H7=16 mm, H8=18 mm. As shown in FIGS. 6G-6J, the total heights of the spacers 600 range from 6 mm to 18 mm (as shown in FIGS. 6G-6J, the height of the spacers 600 increases in 4 mm increments). Also, as shown in embodiments of FIGS. 6G-6J, the cutting line 602 is located 2 mm from the top of the spacers 600. As can be understood by one skilled in the art, the spacers 600 have variable diameters, heights, shapes of the mesh pattern, and thickness.

Additionally, the oval-shaped openings 612 can be aligned in different directions as shown in FIGS. 6A-6D and 6G-6J. Also, some of the openings 612 (or 660) can be circular or any other shape. The openings 612 can have a diameter that varies from the exterior 608 to the interior 606. The oval shaped openings 612 and the circular openings 660 can be arranged in a pattern as illustrated in FIGS. 6A-6D and 6G-6J. Further, one opening 612 can be perpendicularly arranged to the other opening 612. Alternatively, the openings 612 can be arranged at different angles with regard to each other.

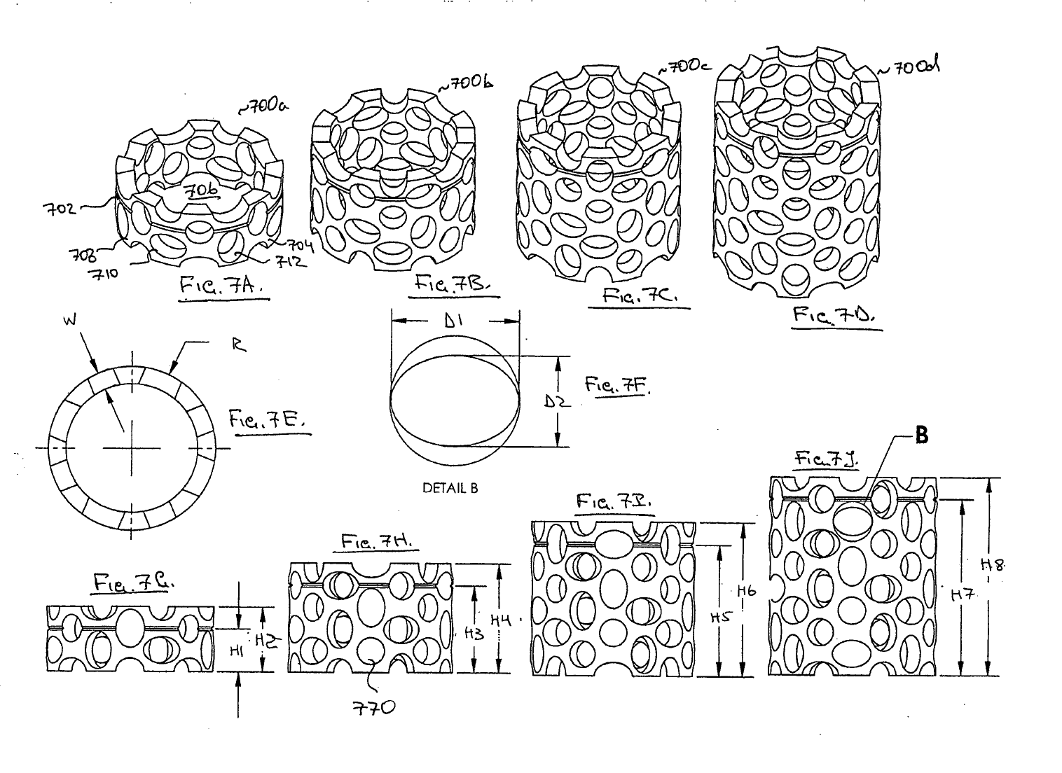

FIGS. 7A-7J illustrate exemplary embodiments of the spacers 700(a, b, c, d) that include a plurality of openings that can be arranged in a mesh pattern. As illustrated in these embodiments, spacers 700 have an outer diameter R. In some embodiments, R=15 mm. As can be understood by one skilled in the art, other diameters of the spacers 700 are possible.

FIGS. 7A-7D are perspective, cross-sectional views of variable height spacers 700 (a, b, c, d). The spacer 700 includes a wall 704 having a thickness W. As shown in of FIG. 7E, which is a top view of the spacers 700, the thickness W can be 1.6 mm. The wall 704 encloses a hollow interior 707 and also includes an exterior 708. Each of the embodiments in FIGS. 7A-7D include an indication cutting line 702 on the exterior 708, where the cutting line 702 is located towards the top of the spacers 700. As can be understood by one skilled in the art, the cutting line 702 can be located anywhere on the outer wall of the spacer 700. Further, there can be more than one indication cutting line on the spacers 700. The cutting line 702 can be configured to allow a surgeon (or other medical personnel, technician, etc.) to adjust the height of the spacer 700 either prior to installation of the spacer 700 or subsequent to installation of the spacer. In some embodiments, the cutting line 702 can be configured to be an indentation in the exterior 708 of the wall 704. The cutting line 702 can be configured to connect openings 712 and 770, as illustrated in FIGS. 7A-7D and 7G-7J. This allows a surgeon (or any other authorized medical personnel) to evenly cut and adjust the spacer 700 to a specific height.

The wall 704 further includes a mesh pattern 710 that consists of variable-shaped openings 710 and 770 that extend from the exterior 708 to the hollow interior 707 of the spacer 700. The opening 712 is shown in more detail in FIG. 7F. In the embodiment of FIGS. 7A-7J, the opening 712 has an oval shape. In this embodiment, the first diameter D1 of the oval shaped opening 712 is approximately 3.5 mm and the second diameter D2 of the oval shaped opening 712 is approximately 2.5 mm. The opening 770 has a round shape with a diameter D3. In some embodiments, D3 is equal to 2.0 mm. FIGS. 7G-7J are side views of variable-height spacers 700. As shown in FIG. 7G, spacer 700a has a total height H1 and a height H2 to the indication cutting line 702. In some embodiments, H1=4 mm, H2=6 mm. As shown in FIG. 7H, spacer 700b has a total height H4 and a height H3 to the indication cutting line 702. In some embodiments, H3=8 mm, H4=10 mm. As shown in FIG. 7I, spacer 700c has a total height H7 and a height H5 to the indication cutting line 702. In some embodiments, H5=12 mm, H6=14 mm. As shown in FIG. 7J, spacer 700d has a total height H8 and a height H7 to the indication cutting line 702. In some embodiments, H7=16 mm, H8=18 mm. As shown in FIGS. 7G-7J, the total heights of the spacers 700 range from 6 mm to 18 mm (as shown in FIGS. 7G-7J, the height of the spacers 700 increases in 4 mm increments). Also, as shown in embodiments of FIGS. 7G-7J, the cutting line 702 is located 2 mm from the top of the spacers 700. As can be understood by one skilled in the art, the spacers 700 have variable diameters, heights, shapes of the mesh pattern, and thickness.

Additionally, the oval-shaped openings 712 can be aligned in different directions as shown in FIGS. 7A-7D and 7G-7J. Also, some of the openings 712 (or 770) can be circular or any other shape. The openings 712 can have a diameter that varies from the exterior 708 to the interior 707. The oval shaped openings 712 and the circular openings 770 can be arranged in a pattern as illustrated in FIGS. 7A-7D and 7G-7J. Further, one opening 712 can be perpendicularly arranged to the other opening 712. Alternatively, the openings 712 can be arranged at different angles with regard to each other.

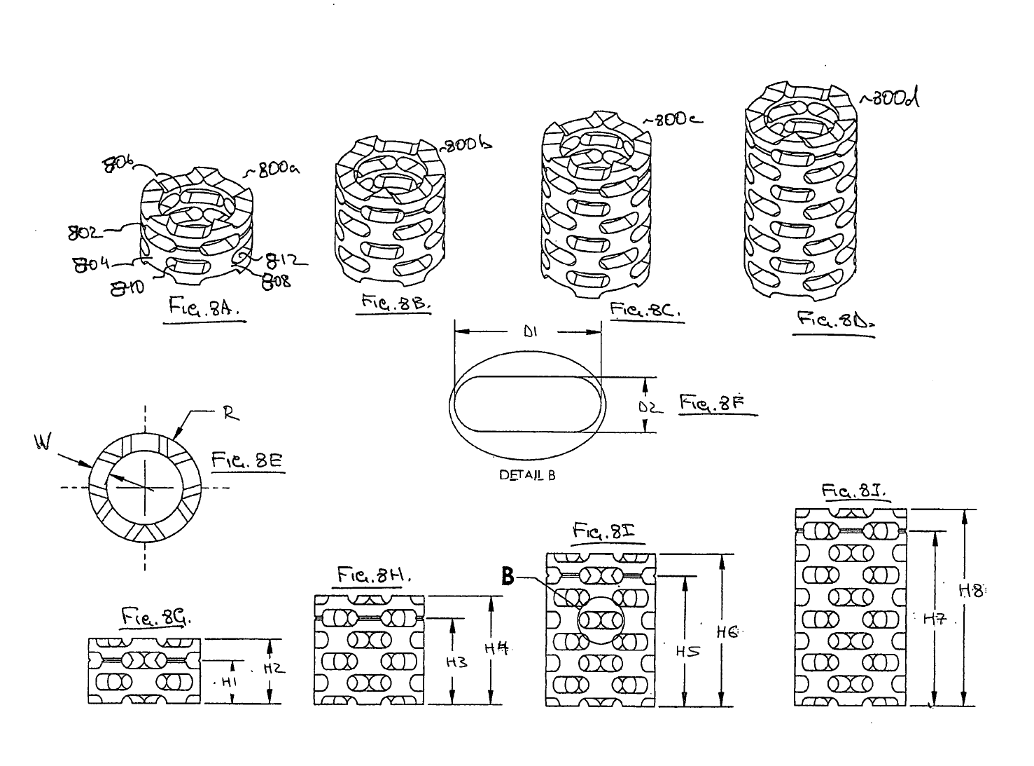

FIGS. 8A-8J illustrate exemplary embodiments of the spacers 800(a, b, c, d) that include a plurality of openings that can be arranged in a mesh pattern. As illustrated in these embodiments, spacers 800 have an outer diameter R. In some embodiments, R=10 mm. As can be understood by one skilled in the art, other diameters of the spacers 800 are possible.

FIGS. 8A-8D are perspective, cross-sectional views of variable height spacers 800 (a, b, c, d). The spacer 800 includes a wall 804 having a thickness W. As shown in of FIG. 8E, which is a top view of the spacers 800, the thickness W can be 1.6 mm. The wall 804 encloses a hollow interior 806 and also includes an exterior 808. Each of the embodiments in FIGS. 8A-8D include an indication cutting line 802 on the exterior 808, where the cutting line 802 is located towards the top of the spacers 800. As can be understood by one skilled in the art, the cutting line 802 can be located anywhere on the outer wall of the spacer 800. Further, there can be more than one indication cutting line on the spacers 800. The cutting line 802 can be configured to allow a surgeon (or other medical personnel, technician, etc.) to adjust the height of the spacer 800 either prior to installation of the spacer 800 or subsequent to installation of the spacer. In some embodiments, the cutting line 802 can be configured to be an indentation in the exterior 808 of the wall 804. The cutting line 802 can be configured to connect openings 812, as illustrated in FIGS. 8A-8D and 8G-8J. This allows a surgeon (or any other authorized medical personnel) to evenly cut and adjust the spacer 800 to a specific height.

The wall 804 further includes a mesh pattern 810 that consists of variable-shaped openings 812 that extend from the exterior 808 to the hollow interior 806 of the spacer 800. The opening 812 is shown in more detail in FIG. 8F. In the embodiment of FIGS. 8A-8J, the opening 812 has an elliptical shape. In this embodiment, the first diameter D1 of the elliptical shape opening 812 is approximately 4.0 mm and the second diameter D2 of the elliptical shape opening 812 is approximately 1.5 mm. FIGS. 8G-8J are side views of variable-height spacers 800. As shown in FIG. 8G, spacer 800a has a total height H2 and a height H1 to the indication cutting line 802. In some embodiments, H1=4 mm, H2=6 mm. As shown in FIG. 8H, spacer 800b has a total height H4 and a height H3 to the indication cutting line 802. In some embodiments, H3=8 mm, H4=10 mm. As shown in FIG. 8I, spacer 800c has a total height H6 and a height H5 to the indication cutting line 802. In some embodiments, H5=12 mm, H6=14 mm. As shown in FIG. 8J, spacer 800d has a total height H8 and a height H7 to the indication cutting line 802. In some embodiments, H7=16 mm, H8=18 mm. As shown in FIGS. 8G-8J, the total heights of the spacers 800 range from 6 mm to 18 mm (as shown in FIGS. 8G-8J, the height of the spacers 800 increases in 4 mm increments). Also, as shown in embodiments of FIGS. 8G-8J, the cutting line 802 is located 2 mm from the top of the spacers 800. As can be understood by one skilled in the art, the spacers 800 have variable diameters, heights, shapes of the mesh pattern, and thickness.

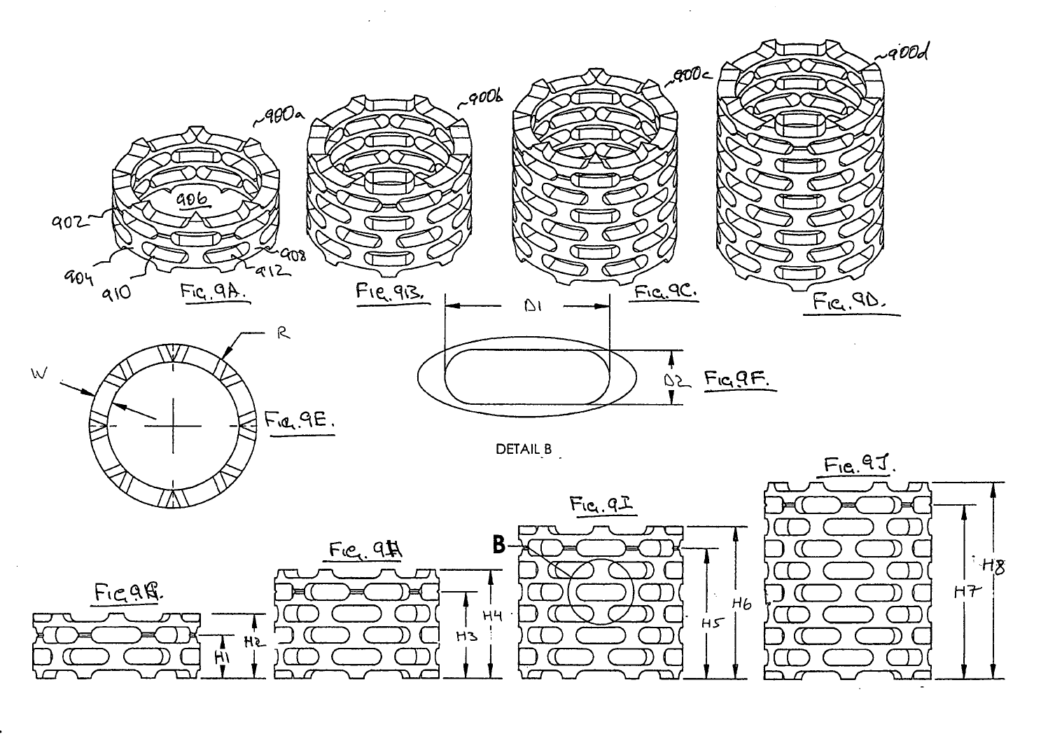

FIGS. 9A-9J illustrate exemplary embodiments of the spacers 900(a, b, c, d) that include a plurality of openings that can be arranged in a mesh pattern. As illustrated in these embodiments, spacers 900 have an outer diameter R. In some embodiments, R=15 mm. As can be understood by one skilled in the art, other diameters of the spacers 900 are possible.

FIGS. 9A-9D are perspective, cross-sectional views of variable height spacers 900 (a, b, c, d). The spacer 900 includes a wall 904 having a thickness W. As shown in of FIG. 9E, which is a top view of the spacers 900, the thickness W can be 1.6 mm. The wall 904 encloses a hollow interior 906 and also includes an exterior 908. Each of the embodiments in FIGS. 9A-9D include an indication cutting line 902 on the exterior 908, where the cutting line 902 is located towards the top of the spacers 900. As can be understood by one skilled in the art, the cutting line 902 can be located anywhere on the outer wall of the spacer 900. Further, there can be more than one indication cutting line on the spacers 900. The cutting line 902 can be configured to allow a surgeon (or other medical personnel, technician, etc.) to adjust the height of the spacer 900 either prior to installation of the spacer 900 or subsequent to installation of the spacer. In some embodiments, the cutting line 902 can be configured to be an indentation in the exterior 908 of the wall 904. The cutting line 902 can be configured to connect openings 912, as illustrated in FIGS. 9A-9D and 9G-9J. This allows a surgeon (or any other authorized medical personnel) to evenly cut and adjust the spacer 900 to a specific height.

The wall 904 further includes a mesh pattern 910 that consists of variable-shaped openings 912 that extend from the exterior 908 to the hollow interior 906 of the spacer 900. The opening 912 is shown in more detail in FIG. 9F. In the embodiment of FIGS. 9A-9J, the opening 912 has an elliptical shape. In this embodiment, the first diameter D1 of the elliptical shape opening 912 is approximately 4.5 mm and the second diameter D2 of the elliptical shape opening 912 is approximately 1.5 mm. FIGS. 9G-9J are side views of variable-height spacers 900. As shown in FIG. 9G, spacer 900a has a total height H2 and a height H1 to the indication cutting line 902. In some embodiments, H1=4 mm, H2=6 mm. As shown in FIG. 9H, spacer 900b has a total height H4 and a height H3 to the indication cutting line 902. In some embodiments, H3=8 mm, H4=10 mm. As shown in FIG. 9I, spacer 900c has a total height H6 and a height H5 to the indication cutting line 902. In some embodiments, H5=12 mm, H6=14 mm. As shown in FIG. 9J, spacer 900d has a total height H8 and a height H7 to the indication cutting line 902. In some embodiments, H7=16 mm, H8=18 mm. As shown in FIGS. 9G-9J, the total heights of the spacers 900 range from 6 mm to 18 mm (as shown in FIGS. 9G-9J, the height of the spacers 900 increases in 4 mm increments). Also, as shown in embodiments of FIGS. 9G-9J, the cutting line 902 is located 2 mm from the top of the spacers 900. As can be understood by one skilled in the art, the spacers 900 have variable diameters, heights, shapes of the mesh pattern, and thickness.

The shape of the openings in the mesh pattern of the spacers can be changed as desired. This can be done with special instruments that are designed to configure the mesh pattern according to the desired shapes. For example, the shape can be changed from a circle to an oval or an “American football” shape. Further, the mesh can also include various shapes or a combination of various shapes, e.g., circles, ovals, polygons, squares, rectangles, ellipses, etc.

As can be understood by one skilled in the art, the thickness W of the wall, the diameter D of the spacer can vary according to a particular design. As can be further understood by one skilled in the art, the diameter D can be configured as an outer diameter of the spacer as illustrated in FIGS. 1A-9J, which means that the diameter D includes the thickness W. In some embodiments, the thickness W can be in the range of 1.0 mm to 2.0 mm. In some embodiments, the range can be from 1.5 mm to 1.7 mm.

Further, the variable openings in the spacers illustrated in the above figures, are configured to allow bone growth once the spacer is installed in the vertebrae (or any other bone structure). This further secures the spacers to the bone matter and provides additional support.

The indication cutting line shown in FIGS. 1A-9J is configured as an indentation in the wall of the spacer. Such indentation can be configured to have a depth in the range from 0.12 mm to 0.24 mm. In some embodiments, the depth can range from 0.15 mm to 0.20 mm. In some embodiments, the depth can be on the order of 0.18 mm.

Additionally, the indication cutting line can be located a distance between 1.0 mm to 4.0 mm from the top of the spacer. In some embodiments, that distance can range from 1.5 mm to 3.5 mm. In yet other embodiments, the distance can range from 2.0 mm to 3.0 mm. Alternatively, the distance can be from 2.0 mm to 2.5 mm. In some embodiments, the distance from the top of the spacer to the indication cutting line can be 2 mm.

In some embodiments, the present invention can be used with the Transforaminal Lumbar Interbody Fusion (“TLIF”) and Posterior Lumbar Interbody Fusion (“PLIF”) instruments for an initial discectomy. Such instruments include Disk Preparation Instruments, such as osteotomes, curettes, shavers, pituitary ronguers, distractors, implant insertion instruments, implant positioning instruments. The spacer can be configured to fit in an anterior portion of the body. In some embodiments, the spacer can be manufactured from titanium alloys, such as, Ti6Al-4V ELI, Ti6Al-4V, Ti6Al-7Nb, CP GRADE 2 TITANIUM and CP GRADE 4 TITANIUM. As can be understood by one skilled in the art, other materials can be used for manufacturing of the spacer.

Example embodiments of the methods and components of the present invention have been described herein. As noted elsewhere, these example embodiments have been described for illustrative purposes only, and are not limiting. Other embodiments are possible and are covered by the invention. Such embodiments will be apparent to persons skilled in the relevant art(s) based on the teachings contained herein. Thus, the breadth and scope of the present invention should not be limited by any of the above-described exemplary embodiments, but should be defined only in accordance with the following claims and their equivalents.

What is claimed:

1. A spacer for use in spine fusion surgical procedures, comprising:
an enclosure having a wall that is configured to enclose a hollow interior;
said wall is further configured to include a plurality of openings spaced throughout said wall;
wherein said openings are configured to connect an exterior of said enclosure to said hollow interior;
said enclosure further includes an indication cutting line configured to allow adjustment of a height of said enclosure.

2. The spacer according to claim 1, wherein said wall includes a thickness in the range of 1.5 mm to 1.7 mm.

3. The spacer according to claim 2, wherein said thickness is about 1.6 millimeters.

4. The spacer according to claim 1, wherein said height of said enclosure is between about 6 millimeters and about 18 millimeters.

5. The spacer according to claim 1, wherein said enclosure includes an outer diameter.

6. The spacer according to claim 1, wherein said diameter is between about 10 millimeters and about 15 millimeters.

7. The spacer according to claim 1, wherein said openings are configured to have a plurality of different shapes;
wherein said shapes are selected from a group consisting of: an oval, an ellipse, a circle, a square, a rectangle, and a polygon.

8. The spacer according to claim 7, wherein said openings are configured to be arranged in a mesh pattern spaced throughout said wall.

9. The spacer according to claim 1, wherein said openings are substantially equally spaced throughout said enclosure.

10. The spacer according to claim 1, wherein said indication cutting line is located in a range of 1 mm to 4 mm from a top of said enclosure.

11. The spacer according to claim 10, wherein said indication cutting line is located about 2 mm from said top of said enclosure.

12. The spacer according to claim 1, wherein the spacer is configured to be used with Transforaminal Lumbar Interbody Fusion (“TLIF”) instruments.

13. The spacer according to claim 1, wherein the spacer is configured to be used with Posterior Lumbar Interbody Fusion (“PLIF”) instruments.

14. The spacer according to claim 1, wherein the spacer is manufactured from a material selected from a group consisting of titanium alloys, Ti6Al-4V ELI, Ti6Al-4V, Ti6Al-7Nb, CP GRADE 2 TITANIUM and CP GRADE 4 TITANIUM.

15. The spacer according to claim 1, wherein said indication cutting line is configured as an indentation in said wall, wherein said indentation has a depth in the range of 0.12 to 0.24 mm.

16. The spacer according to claim 15, wherein said indication cutting line is configured as an indentation in said wall, wherein said indentation has a depth of 0.18 mm.

17. The spacer according to claim 1, further comprising a plurality of indication cutting lines.

18. The spacer according to claim 1, wherein said adjustment is configured to shorten said height of said enclosure.

19. A spinal vertebral replacement assembly, comprising:
a spacer having:
an enclosure having a wall that is configured to enclose a hollow interior;
said wall is further configured to include a plurality of openings spaced throughout said wall;
wherein said openings are configured to connect an exterior of said enclosure to said hollow interior;
said enclosure further includes an indication cutting line configured to allow adjustment of a height of said enclosure.
